(19) United States
(12) Patent Application Publication
(10) Pub. No.: US 2026/0258190 A1
(43) Pub. Date: Sep. 3, 2026

(54) POLYISOCYANATES WITH IMPROVED PROPERTIES

(71) Applicant: COVESTRO DEUTSCHLAND AG, Leverkusen (DE)

(72) Inventor: Frank Richter, Leverkusen (DE)

(21) Appl. No.: 19/154,015

(22) PCT Filed: Feb. 6, 2024
(86) PCT No.: PCT/EP2024/052830

§ 371 Date: Aug. 6, 2025

(30) Foreign Application Priority Data
Feb. 9, 2023 (EP) 23155763.8

(51) Int. Cl. C08G 18/79; C08G 18/02; C08G 18/20

(52) U.S. Cl. CPC C08G 18/792; C08G 18/022; C08G 18/2018; C08G 18/2081

(57) ABSTRACT

The invention relates to a process for producing modified isocyanates by reacting at least one organic di- and/or triisocyanate in the at least temporary absence of a component A1 comprising at least one tetramethyl ammonium salt and/or at least one cyclic ammonium salt containing a cation of formula (I), wherein: Y is a linear or branched C₂-C₂₀ segment which optionally also carries other substituents and is optionally interrupted by heteroatoms from the group including oxygen, sulphur, nitrogen and aromatic rings, and which optionally also carries other rings; and wherein either the substituents R¹ and R² in the N position, independently of each other, are the same or different, substituted or unsubstituted, optionally branched, aliphatic C₁-C₂₀ groups, aromatic C₆-C₂₀ groups or araliphatic C₇-C₂₀ groups; or the substituents R¹ and R² in the N position together form a ring segment X, with the same definition as given above for Y or a different definition; and when a predefinable degree of conversion based on the total amount of NCO groups of the at least one organic di- and/or triisocyanate is reached, stopping the reaction by adding a substoichiometric amount, based on the molar amount of the cation of formula (I) in component A1, of a component A2 comprising at least one acidic compound which has a pKₛ value less than 4.0 and is other than HF; and, after optional purification, the resulting modified isocyanate is mixed with a component A3 comprising an acidic compound which has a pKₛ value less than 4.0 and is other than HF, and the at least one acidic compound in components A2 and A3 can be the same or different.

The invention relates to polyisocyanates having improved properties such as freedom from odor, extremely low monomer content, good monomer stability and a low propensity for clouding. The invention also relates to a process for producing these polyisocyanates having improved properties themselves, to the use thereof for producing polyurethane bodies or coatings and to the polyurethane bodies or coatings. The invention further relates to one- or two-component systems comprising the polyisocyanates having improved properties.

The oligomerization or polymerization of isocyanates, especially to form higher molecular weight oligomer mixtures having uretdione (“dimer”), isocyanurate (“trimer”) and/or iminooxadiazinedione structures (“asymmetric trimer”) in the molecular structure, has long been known. As can be seen above, the oligomerization and polymerization of isocyanates are based in principle on the same chemical reactions. The reaction of a relatively small number of isocyanates with one another is referred to as oligomerization. The reaction of a relatively large number of isocyanates is referred to as polymerization. In the context of the present invention, the oligomerization or polymerization of isocyanates described above is referred to collectively as isocyanate modification or modification of isocyanates.

The modified polyisocyanates comprising free NCO groups, which optionally may also have been temporarily deactivated with blocking agents, are exceptionally high-quality starting materials for the production of a multiplicity of polyurethane plastics and coating compositions.

A series of industrial methods for isocyanate modification have been established in which the isocyanate to be modified, usually a diisocyanate, is generally reacted by addition of catalysts and these are then rendered inactive (deactivated) by suitable measures, when the desired degree of conversion of the isocyanate to be modified has been reached, and the polyisocyanate obtained is generally separated from the unreacted monomer. A summary of these processes from the prior art may be found in H. J. Laas et al., J. Prakt. Chem. 1994, 336, 185 ff.

Compounds of ionic composition have proven to be effective as modification catalysts since they may be used in very low quantities, relative to the monomer to be converted, and lead extremely rapidly to the desired result, the cations being important in particular from the aspect of solubility of the respective salt in the isocyanate medium.

WO 2015/124504 A1 and WO 2017/029266 A1 describe very stable ammonium salts in which the charge-bearing nitrogen atom forms part of a ring system. However, these compounds have the disadvantage of being poorly soluble in the isocyanate-functional polyisocyanate resin and therefore being capable of causing clouding in the final process products.

According to the teaching of WO 2021/122508 A1 this problem is avoided through incorporation of hydroxy functions into the catalyst molecule. However, the special hydroxy-functional ammonium salts have the disadvantage that they are not commercially available and that their synthesis is very costly and complex. Inexpensive hydroxy-functional ammonium salts in which the hydroxy function is not bonded to a carbon atom of the charge-carrying, nitrogen-containing cycle do not exhibit any advantages in the context of the present invention: they are neither free from odors, nor are the monomers that are generated in the process and typically recycled free from disruptive catalyst decomposition products.

It was accordingly an object of the invention to provide polyisocyanates having improved properties such as freedom from odor, an extremely low monomer content of <0.1% by weight, high retrocleavage stability and a low propensity for clouding. It was especially an object of the invention to provide polyisocyanates having a high content of iminooxadiazinedione structures and the aforementioned improved properties.

In light of this need a first subject of the present invention relates to a process for producing modified isocyanates where at least one organic di- and/or triisocyanate is reacted in at least the temporary presence of a component A1 comprising at least one tetramethylammonium salt and/or at least one cyclic ammonium salt having a cation of formula I

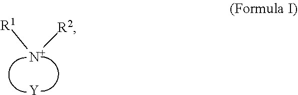

wherein
Y is a linear or branched C₂-C₂₀ segment optionally bearing further substituents and optionally interrupted by heteroatoms from the group of oxygen, sulfur, nitrogen and aromatic rings and optionally comprising further rings and
the nitrogen substituents R¹ and R² either independently represent identical or different, substituted or unsubstituted, optionally branched aliphatic C₁-C₂₀ radicals, aromatic C₆-C₂₀ radicals or araliphatic C₇-C₂₀ radicals or
the nitrogen substituents R¹ and R² together form a ring segment X for which the same or a different definition for Y mentioned above applies, the reaction is terminated upon achieving a predeterminable degree of conversion based on the total amount of NCO groups of the at least one organic di- and/or triisocyanate by addition of a substoichiometric amount, based on the molar amount of the cation of formula I in component A1, of a component A2 comprising at least one acidic compound which has a pKₐ value below 4.0 and is distinct from HF and the obtained modified isocyanate (optionally after further purification) is admixed with a component A3 comprising at least one acidic compound which has a pKₐ value below 4.0 and is distinct from HF, wherein the at least one acidic compound in component A2 and A3 may be distinct from one another or identical.

Also in light of this need one subject of the invention relates to a catalyst kit for isocyanate modification comprising three separate components A1, A2 and A3, wherein
a) component A1 contains at least one tetramethylammonium salt and/or at least one cyclic ammonium salt having a cation of formula I,

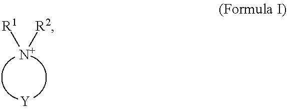

wherein
Y is a linear or branched C₂-C₂₀ segment optionally bearing further substituents and optionally interrupted by heteroatoms from the group of oxygen, sulfur, nitrogen and aromatic rings and optionally comprising further rings and
the nitrogen substituents R¹ and R² either independently represent identical or different, substituted or unsubstituted, optionally branched aliphatic C₁-C₂₀ radicals, aromatic C₆-C₂₀ radicals or araliphatic C₇-C₂₀ radicals or
the nitrogen substituents R¹ and R² together form a ring segment X for which the same or a different definition for Y mentioned above applies;
b) component A2 is employed in a substoichiometric amount based on the molar amount of the cation of formula I in component A1 and comprises or consists of at least one acidic compound which has a pKₐ value below 4.0 and is distinct from HF;
c) component A3 contains at least one acidic compound which has a pKₐ value below 4.0 and is distinct from HF, wherein the at least one acidic compound in component A2 and A3 may be identical or different from one another.

Also in light of this need one subject of the invention relates to a use of at least one catalyst kit according to the invention for modifying isocyanates and for preventing clouding in the modified isocyanates.

The references to “comprising”, “containing”, etc. preferably denote “substantially consisting of” and very particularly preferably denote “consisting of”. The further embodiments recited in the claims and in the description may be combined as desired, especially also among the different subjects according to the invention, provided that the opposite is not clearly apparent from the context.

“At least one”, as used herein, refers to 1 or more, for example 2, 3, 4, 5, 6, 7, 8, 9 or more. In connection with constituents of the compounds described herein, this figure refers not to the absolute number of molecules, but rather to the nature of the constituent. “At least one cyclic ammonium salt” is therefore to be understood as meaning for example that only one type of cyclic ammonium salt or two or more different types of cyclic ammonium salts may be present without specifying the amount of the individual compounds.

Numerical values specified herein without decimal places refer in each case to the full value specified to one decimal place. For example, “99%” signifies “99.0%”.

Numerical ranges given in the format “of/from x to y” include the recited values. If multiple preferred numerical ranges are specified in this format it goes without saying that all ranges formed by the combination of the different end points are likewise captured.

For polybasic acids the pKₐ value is in the present case considered to be the lowest value, for example for phosphoric acid it is pKa1 of 2.16 and not the further values pKa2=7.20 and pKa3=12.33.

The term “aliphatic” is presently defined as meaning non-aromatic hydrocarbon groups that are saturated or unsaturated.

The term “araliphatic” is presently defined as meaning hydrocarbon radicals consisting of both an aromatic hydrocarbon group and a saturated or unsaturated hydrocarbon group which is bonded directly to the aromatic radical.

The term “alicyclic” or “cycloaliphatic” is presently defined as meaning optionally substituted carbocyclic or heterocyclic compounds or units which are not aromatic (for example cycloalkanes, cycloalkenes or oxa-, thia-, aza- or thiazacycloalkanes). Particular examples are cyclohexyl groups, cyclopentyl groups and their N- or O-heterocyclic derivatives such as for example pyrimidine, pyrazine, tetrahydropyran or tetrahydrofuran.

In the event that the groups or compounds are disclosed as “optionally substituted” or “substituted”, suitable substituents are —F, —Cl, —Br, —I, —OCH3, —OCH2CH3, —O-isopropyl or —O-n-propyl, —OCF3, —CF3, —S—C1-6-alkyl and/or (optionally via a pendant heteroatom) a linear or branched aliphatic and/or alicyclic structural unit having 1 to 12 carbon atoms which in each case functions as a substitute for a carbon-bonded hydrogen atom of the respective molecule. Preferred substituents are halogen (especially —F, —Cl), C₁-C₆-alkoxy (especially methoxy and ethoxy), trifluoromethyl and trifluoromethoxy which in each case function as a substitute for a carbon-bonded hydrogen atom of the respective molecule.

According to the invention component A1 contains at least one tetramethylammonium salt and/or at least one cyclic ammonium salt having a cation of formula I, wherein the cyclic ammonium salt having a cation of formula I is preferred.

In a first preferred embodiment Y represents an alkylene chain segment which comprises four to seven members together with the charge-carrying nitrogen atom and optionally bears further substituents.

In a likewise preferred embodiment, R¹ and R² together with the charge-carrying nitrogen atom represent a ring segment X identical to Y or different from Y, wherein X is a C₄-C₆-alkylene chain segment optionally bearing further substituents and may optionally bear further substituents.

In a further preferred embodiment segment Y and/or ring segment X have a linear structure.

Cations of formula I, in which the nitrogen substituents R¹ and R² together form a ring system or the ring segment X, are spirocyclic compounds. The latter are easily obtainable by reaction of secondary amines in which the nitrogen atom is part of a ring system with suitably substituted dihaloalkanes and subsequent anion exchange. A preferred route to their efficient synthesis is apparent from example 1.

X and Y in formula I may preferably independently represent optionally substituted alkylene groups, wherein preference is given to C₄-C₆-alkylene chains especially in both N-centered rings. The C₄-C₆-alkylene chains preferably have a linear structure. These are easily obtainable for example by reaction of optionally C-substituted pyrrolidines, piperidines and azepanes (1H-hexahydroazepines) with optionally substituted 1,4-, 1,5- and 1,6-dihaloalkanes, wherein halogen represents Cl, Br and I, preferably Cl.

In addition, for example, by analogous reaction of optionally C-substituted oxazolidines, isoxazolidines, oxazinanes, morpholines and oxazepanes and the analogs of the aforementioned N—O heterocycles which contain S rather than O, and also imidazolidines, pyrazolidines, piperazines and structurally related compounds, with the abovementioned dihaloalkanes, it is also possible to obtain representatives having C chains interrupted by heteroatoms in one of the X or Y segments of the general formula I. In the case of species containing 2 or more nitrogen atoms, it is additionally possible, by appropriate variation of the reaction conditions, also to produce salts having a doubly or multiply charged cation or, by prior suitable substitution of the nitrogen atom(s), to arrive at singly positively charged cations of formula I in which one or more exocyclic alkyl substituent(s) is/are present on the trivalent nitrogen atom(s) of the ring X or Y.

It goes without saying that it is also possible through suitable choice of the alkylating agent to introduce a structural variation into the ring segment X or Y; examples include reactions of alpha-omega-dihaloalkyl ethers with the aforementioned secondary amines.

In a further preferred embodiment, R¹ and R² independently represent identical or different C₁-C₈-alkyl substituents or identical or different benzyl radicals optionally substituted at the aromatic ring, preferably identical or different C₁-C₆-alkyl substituents and particularly preferably identical or different C₁-C₆-alkyl substituents having a linear structure and very particularly preferably methyl.

Anions used in the compounds of formula I may in principle be any structure type known to be catalytically active with respect to isocyanates, preference being given to hydroxide, alkanoate, carboxylate, heterocycles having at least one negatively charged nitrogen atom in the ring, especially azolate, imidazolate, triazolate, tetrazolate, fluoride, hydrogendifluoride, higher polyfluorides or mixtures of these (adducts of more than one equivalent of HF onto compounds containing fluoride ions), wherein according to the invention fluorides, hydrogendifluorides and higher polyfluorides lead to products having a high iminooxadiazinedione group content.

The catalysts of component A1 employable according to the invention may be used individually or in any desired mixtures with one another. For instance, the solutions of quaternary ammonium hydroxides in various alcohols, depending on the pKa value of the base and of the alcohol used, are present partially or completely as ammonium salts having an alkoxide anion. This equilibrium can be shifted wholly to the side of complete alkoxide formation by removing the water of reaction resulting from this reaction.

In the process according to the invention, it may further be provided that the oligomerization be conducted in the presence of a solvent.

For performance of the process according to the invention, it is possible in principle to use any known mono-, di- or polyisocyanates from the prior art, individually or in any desired mixtures with one another.

Examples include: pentamethylene diisocyanate (PDI), hexamethylene diisocyanate (HDI), 2-methylpentane 1,5-diisocyanate (MPDI), 2,4,4-trimethylhexane 1,6-diisocyanate and 2,2,4-trimethylhexane 1,6-diisocyanate (TMDI), 4-isocyanatomethyloctane 1,8-diisocyanate (nonane triisocyanate, NTI), 3(4)-isocyanatomethyl-1-methylcyclohexyl isocyanate (IMCI), isophorone diisocyanate (IPDI), 1,3- and 1,4-bis(isocyanatomethyl)benzene (XDI), 1,3- and 1,4-bis(isocyanatomethyl)cyclohexane (H₆XDI), norbornane diisocyanate (NBDI), tolylene 2,4- and 2,6-diisocyanate (TDI), bis(4-isocyanatophenyl)methane (4,4′MDI), 4-isocyanatophenyl-2-isocyanatophenylmethane (2,4′MDI) and polycyclic products obtainable by formaldehyde-aniline polycondensation and subsequent conversion of the resulting (poly)amines to the corresponding (poly)isocyanates (polymer MDI).

Preference is given to aromatic diisocyanates, i.e. diisocyanates in which both NCO groups are bonded to an sp²-hybridized carbon atom, or aliphatic diisocyanates, i.e. diisocyanates in which both NCO groups are bonded to an sp³-hybridized carbon atom.

Particular preference is given to PDI, HDI, MPDI, TMDI, NTI, IPDI, IMCI, XDI, H₆XDI, MDI, or NBDI and very particular preference is given to pentamethylene diisocyanate (PDI), hexamethylene diisocyanate (HDI), isophorone diisocyanate (IPDI), 1,3- and 1,4-bis(isocyanatomethyl)cyclohexane (HXDI) and/or norbornane diisocyanate (NBDI).

It is immaterial by which processes the abovementioned isocyanates are generated, i.e. with or without use of phosgene.

The amount of the catalyst of component A1 employable in the process according to the invention is guided primarily by the organic isocyanate used and the desired reaction rate and is preferably between ≥0.001 and ≤5 mol %, preferably between ≥0.002 and ≤2 mol %, based on the sum of the molar amounts of the employed isocyanate and of the catalyst.

In the process according to the invention or in the catalyst kit according to the invention, the catalyst may be employed undiluted or dissolved in solvents. Useful solvents here include all compounds which do not react with the catalyst and are capable of dissolving it to a sufficient degree, for example optionally halogenated aliphatic or aromatic hydrocarbons, alcohols, ketones, esters and ethers. Preference is given to using alcohols.

The process according to the invention may be performed in the temperature range from 0° C. to +250° C., preferably 20° C. to 200° C., particularly preferably 40° C. to 150° C., and can be interrupted at any degrees of conversion, preferably once 5% to 80%, particularly preferably 10% to 60%, of the isocyanate used has been converted.

Stopping of the reaction (hereinbelow also referred to as deactivation of the catalyst or termination of the catalyzed isocyanate oligomerization) may be achieved by any desired methods, wherein it is essential to the invention that a chemical deactivation of the catalyst is carried out in at least two steps to arrive at the products according to the invention having a low propensity for crystallization and a low residual monomer content.

According to the invention termination of the catalyzed isocyanate oligomerization is therefore carried out by employing an amount of at least one catalyst poison (hereinbelow also referred to as “stopper” and collectively described as component A2) which is substoichiometric relative to the employed catalyst but sufficient for termination of the catalyzed reaction and adding a second dose as component A3 of the same or a different catalyst poison to the product having a low residual monomer content according to the invention after the, preferably distillative, monomer separation.

The amount of component A3 added as the second dose (hereinbelow also referred to as “stabilizer”) may be freely chosen over a wide range and is preferably added in a molar amount which is stoichiometric, particularly preferably superstoichiometric, based on the calculated molar amount of the cation of formula I in component A1 remaining after subtraction of the amount already present as component A2. The term “superstoichiometric” is preferably to be understood as meaning that not more than double the molar amount of catalyst poison relative to the molar amount of the cation of formula I need be added to component A1.

Only this measure according to the invention makes it possible to generate odor-neutral, crystallization-stable products having an extremely low monomer content and high retrocleavage stability for a long period and also at low temperatures and/or when using typical lacquer solvents for the polyisocyanate resin. The process according to the invention moreover also benefits from a significantly improved process stability through more uniform catalyst consumption without drifts and a lower propensity for caking, clouding, and solid separation etc in the plants.

Suitable stoppers generally include acidic compounds which have the pKₐ values defined in the present text and are distinct from HF, for example alkanesulfonic and arylsulfonic acids such as for example naphthalene monosulfonic and disulfonic acids, toluenesulfonic acid, dodecylbenzenesulfonic acid, methanesulfonic acid, phosphoric acid and acidic esters of phosphoric acid such as for example dibutyl phosphate and/or monobutyl phosphate and any desired mixtures of the aforementioned compounds.

In a preferred embodiment the at least one acidic compound of component A2 and/or is selected from the group comprising or consisting of alkanesulfonic and arylsulfonic acids, for example naphthalenemonosulfonic and naphthalenedisulfonic acids, toluenesulfonic acid, dodecylbenzenesulfonic acid, methanesulfonic acid, phosphoric acid and acidic esters of phosphoric acid, for example dibutyl phosphate and/or monobutyl phosphate and any desired mixtures of the aforementioned compounds, preferably of aromatic sulfonic acids and particularly preferably of dodecylbenzenesulfonic acid and toluenesulfonic acid.

In a further preferred embodiment, unconverted organic isocyanate is removed after deactivation of the catalyst system by any desired process from the prior art, for example by (thin film) distillation or extraction, and preferably subsequently reused (recycled).

Irrespective of the anion responsible for catalytic activity and selectivity, the catalysts according to the invention comprising cyclic segments are generally substantially more stable in the organic isocyanate to be reacted than the prior art open-chain derivatives known from the literature, though this can lead to disadvantages in industrial performance due to poor solubility and clouding of the catalyst consequent products in the polyisocyanate, as well as entailing advantages however through reduced contamination of the recyclate monomer by N-containing cleavage products from the decomposition of the cation.

In a particular continuously operating embodiment of the process according to the invention, the oligomerization may be undertaken continuously, for example in a tubular reactor.

The modification process according to the invention very generally makes it possible to obtain a wide range of modified isocyanates which are of high quality and therefore very valuable to the polyurethane sector. The present invention further provides a modified isocyanate obtainable or produced by the process according to the invention.

Depending on the starting (di)isocyanate used and the reaction conditions, the process according to the invention affords polyisocyanates of what is known as the isocyanate trimer type (i.e. containing isocyanurate and/or iminooxadiazinedione structures) having a low proportion of uretdione groups (“isocyanate dimers”). The proportion of the latter in the process products generally increases with increasing reaction temperature.

Preference is given to polyisocyanates having a high iminooxadiazinedione content which according to the invention are obtainable by polyfluoride catalysis. The term “high iminooxadiazinedione content” is to be understood as meaning at least 30 mol %, preferably >35 mol % and particularly preferably >40 mol % based on the sum of isocyanurate and iminooxadiazinedione groups. The aforementioned molar ratios may be determined for example by NMR spectroscopy (see example section).

The products or product mixtures obtainable by the process according to the invention are consequently versatile starting materials for production of optionally foamed plastic(s) and of lacquers, coating compositions, adhesives and additives. Therefore, the present invention further provides for the use of the modified isocyanates according to the invention for the production of foamed or unfoamed plastics and lacquers, coating compositions, adhesives and additives.

Consequently, the present invention further provides polyurethane bodies obtainable or produced by reacting at least one monomeric diisocyanate and/or polyisocyanate with at least one polyol component in the presence of the catalyst component according to the invention. Where foamed polyurethane bodies are concerned, preference is given to PIR foams.

The process products according to the invention may be used as such or in conjunction with other prior art isocyanate derivatives, for example polyisocyanates containing uretdione, biuret, allophanate, isocyanurate and/or urethane groups whose free NCO groups have optionally been deactivated with blocking agents.

The present invention further provides a one- or two-component system containing a component A) comprising at least one modified isocyanate according to the invention and a component B) comprising at least one NCO-reactive compound and a coating obtainable or produced by curing a one- or two-component system according to the invention optionally with heating and/or in the presence of a catalyst but also the substrates coated with at least one one- or two-component system according to the invention which has optionally been cured with heating.

Even just the surprising observation that the modified isocyanates according to the invention do not exhibit delayed-onset clouding indicates that the products according to the invention must be distinct from the prior art in terms of their structure and composition. The isocyanates modified according to the invention differ by the cation (from component A1) to anion (from components A2 and A3) ratios, more precisely the cation (from A1) to halide ratio, preferably the cation (from A1) to chloride and/or cation (from A1) to bromide ratio and the cation (from A1) to anion (from A2 and/or A3) sulfonate ratio. Especially the cation (from A1) to anion (from A2 and/or A3) ratio, wherein the anions resulting from A2 and/or A3 contain phosphorus and/or sulfur, preferably sulfur. The differences are reflected in the cured or formed polyurethane bodies and thus a composite component comprising a material that is at least partly bonded to a polyurethane body according to the invention or a coating according to the invention is likewise a subject of the invention.

In the present case, the term “modified isocyanate” has the meaning defined at the outset and preferably represents a polyisocyanate having a statistical average of at least 1.5 NCO groups. The modified isocyanate according to the invention is synonymous with a modified isocyanate composition since, for example, the cations and anions are not separably present.

The present invention relates in particular to the following embodiments:

In a first embodiment the invention relates to a process for producing modified isocyanates where at least one organic di- and/or triisocyanate is reacted in at least the temporary presence of a component A1 comprising at least one tetramethylammonium salt and/or at least one cyclic ammonium salt having a cation of formula I

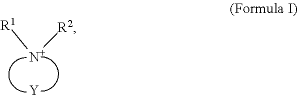

wherein
Y is a linear or branched C₂-C₂₀ segment optionally bearing further substituents and optionally interrupted by heteroatoms from the group of oxygen, sulfur, nitrogen and aromatic rings and optionally comprising further rings and
the nitrogen substituents R¹ and R² either independently represent identical or different, substituted or unsubstituted, optionally branched aliphatic C₁-C₂₀ radicals, aromatic C₆-C₂₀ radicals or araliphatic C₇-C₂₀ radicals or
the nitrogen substituents R¹ and R² together form a ring segment X for which the same or a different definition for Y mentioned above applies,
   the reaction is terminated upon achieving a predeterminable degree of conversion based on the total amount of NCO groups of the at least one organic di- and/or triisocyanate by addition of a substoichiometric amount, based on the molar amount of the cation of formula I in component A1, of a component A2 comprising at least one acidic compound which has a pKₐ value below 4.0 and is distinct from HF
   and the obtained modified isocyanate (after optional purification) is admixed with a component A3 comprising at least one acidic compound which has a pKₐ value below 4.0 and is distinct from HF, wherein the at least one acidic compound in component A2 and A3 may be distinct from one another or identical.

In a second embodiment the invention relates to a process for producing modified isocyanates according to embodiment 1, characterized in that R¹ and R² in the cation of formula I independently represent identical or different C₁-C₈-alkyl substituents or identical or different benzyl radicals optionally substituted at the aromatic ring, preferably identical or different C₁-C₆-alkyl substituents, particularly preferably identical or different C₁-C₆-alkyl substituents having a linear structure and very particularly preferably methyl, or that R¹ and R² together with the charge-carrying nitrogen atom represent a ring segment X identical to Y or different from Y, wherein X is a C₄-C₆-alkylene chain segment optionally bearing further substituents and may optionally bear further substituents.

In a third embodiment the invention relates to a process for producing modified isocyanates according to embodiment 1 or 2, characterized in that Y in the cation of formula I represents an alkylene chain segment which comprises four to seven members together with the charge-carrying nitrogen atom and optionally bears further substituents, preferably segment Y and/or ring segment X have a linear structure.

In a fourth embodiment the invention relates to a process for producing modified isocyanates according to any of the preceding embodiments, characterized in that component A1 contains an anion selected from the group consisting of hydroxide, alkanoate, carboxylate, heterocycles having at least one negatively charged nitrogen atom in the ring, fluoride, hydrogendifluoride, higher polyfluorides, adducts of more than one equivalent of HF onto compounds containing fluoride ions and any desired mixtures thereof.

In a fifth embodiment the invention relates to a process for producing modified isocyanates according to any of the preceding embodiments, characterized in that the at least one acidic compound in component A1 and/or A2 has a pKₐ value below 2.0.

In a sixth embodiment the invention relates to a process for producing modified isocyanates according to any of the preceding embodiments, characterized in that component A3 (stabilizer) is added in a molar amount which together with component A2 (stopper) is stoichiometric, preferably superstoichiometric, based on the amount of the cation of formula I in component A1.

In a seventh embodiment the invention relates to a process for producing modified isocyanates according to any of the preceding embodiments, characterized in that the at least one acidic compound of component A2 and/or A3 is selected from the group comprising or consisting of alkanesulfonic and arylsulfonic acids, for example naphthalenemonosulfonic and naphthalenedisulfonic acids, toluenesulfonic acid, dodecylbenzenesulfonic acid, methanesulfonic acid, phosphoric acid and acidic esters of phosphoric acid, for example dibutyl phosphate and/or monobutyl phosphate and any desired mixtures of the aforementioned compounds, preferably of aromatic sulfonic acids and particularly preferably of dodecylbenzenesulfonic acid and toluenesulfonic acid.

In an eighth embodiment the invention relates to a process for producing modified isocyanates according to any of the preceding embodiments, characterized in that the organic diisocyanate is selected from the group comprising or consisting of PDI, HDI, MPDI, TMDI, NTI, IPDI, IMCI, XDI, H6XDI, MDI, TDI or NBDI, preferably from the group comprising or consisting of pentamethylene diisocyanate, hexamethylene diisocyanate, isophorone diisocyanate, 1,3- and 1,4-bis(isocyanatomethyl)cyclohexane and/or norbornane diisocyanate.

In a ninth embodiment the invention relates to a catalyst kit for isocyanate modification comprising three components A1, A2 and A3, wherein
a) component A1 contains at least one tetramethylammonium salt and/or at least one cyclic ammonium salt having a cation of formula I,

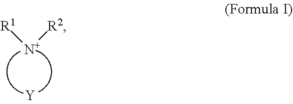

wherein
Y is a linear or branched C₂-C₂₀ segment optionally bearing further substituents and optionally interrupted by heteroatoms from the group of oxygen, sulfur, nitrogen and aromatic rings and optionally comprising further rings and
the nitrogen substituents R¹ and R² either independently represent identical or different, substituted or unsubstituted, optionally branched aliphatic C₁-C₂₀ radicals, aromatic C₆-C₂₀ radicals or araliphatic C₇-C₂₀ radicals or
the nitrogen substituents R¹ and R² together form a ring segment X for which the same or a different definition for Y mentioned above applies;
b) component A2 comprises or consists of at least one acidic compound which has a pKₐ value below 4.0 and is distinct from HF and is employed in a substoichiometric amount based on the molar amount of the cation of formula I in component A1; and
c) component A3 contains at least one acidic compound which has a pKₐ value below 4.0 and is distinct from HF, wherein the at least one acidic compound in component A2 and A3 may be identical or different from one another.

In a tenth embodiment the invention relates to a catalyst kit for isocyanate modification according to embodiment nine, characterized in that R¹ and R² in the cation of formula I independently represent identical or different C₁-C₈-alkyl substituents or identical or different benzyl radicals optionally substituted at the aromatic ring, preferably identical or different C₁-C₆-alkyl substituents and particularly preferably identical or different C₁-C₆-alkyl substituents having a linear structure, or that R¹ and R² together with the charge-carrying nitrogen atom represent a ring segment X identical to Y or different from Y, wherein X is a C₄-C₆-alkylene chain segment optionally bearing further substituents and may optionally bear further substituents.

In an eleventh embodiment the invention relates to a catalyst kit for isocyanate modification according to either of embodiments nine or ten, characterized in that Y in the cation of formula I represents an alkylene chain segment which comprises four to seven members together with the charge-carrying nitrogen atom and optionally bears further substituents, preferably segment Y and/or ring segment X have a linear structure.

In a twelfth embodiment the invention relates to a catalyst kit for isocyanate modification according to any of embodiments nine to eleven, characterized in that component A1 contains an anion selected from the group consisting of hydroxide, alkanoate, carboxylate, heterocycles having at least one negatively charged nitrogen atom in the ring, fluoride, hydrogendifluoride, higher polyfluorides, adducts of more than one equivalent of HF onto compounds containing fluoride ions and any desired mixtures thereof.

In a thirteenth embodiment the invention relates to a catalyst kit for isocyanate modification according to any of embodiments nine to twelve, characterized in that the at least one acidic compound in component A1 and/or A2 has a pKₐ value below 2.0.

In a fourteenth embodiment the invention relates to a catalyst kit for isocyanate modification according to any of embodiments nine to thirteen, characterized in that component A3 (stabilizer) is added in a molar amount which together with component A2 (stopper) is stoichiometric, preferably superstoichiometric, based on the amount of the cation of formula I in component A1.

In a fifteenth embodiment the invention relates to a catalyst kit for isocyanate modification according to any of embodiments nine to fourteen, characterized in that the at least one acidic compound of component A2 and/or A3 is selected from the group comprising or consisting of alkanesulfonic and arylsulfonic acids, for example naphthalenemonosulfonic and naphthalenedisulfonic acids, toluenesulfonic acid, dodecylbenzenesulfonic acid, methanesulfonic acid, phosphoric acid and acidic esters of phosphoric acid, for example dibutyl phosphate and/or monobutyl phosphate and any desired mixtures of the aforementioned compounds, preferably of aromatic sulfonic acids and particularly preferably of dodecylbenzenesulfonic acid and toluenesulfonic acid.

In a sixteenth embodiment the invention relates to a use of at least the catalyst kit according to any of embodiments nine to fifteen in isocyanate modification to prevent clouding in the modified isocyanate.

In a seventeenth embodiment the invention relates to a modified isocyanate, obtainable or produced, preferably directly obtainable by a process according to any of embodiments one to eight.

In an eighteenth embodiment the invention relates to a one-component system containing a modified isocyanate according to embodiment seventeen in which the NCO groups are blocked or a two-component system comprising a component 1) comprising at least one modified isocyanate according to embodiment seventeen and a component 2) comprising at least one NCO-reactive compound.

In a nineteenth embodiment the invention relates to a coating obtainable or produced by applying a one- or two-component system according to embodiment eighteen to a substrate and curing, optionally with heating and/or in the presence of a catalyst.

In a twentieth embodiment the invention relates to a composite component comprising a material that is at least partly bonded to a coating according to embodiment nineteen.

The comparative examples and examples which follow are intended to further illustrate the invention without limiting it.

### EXAMPLES

All percentages, unless noted otherwise, are to be understood as meaning percent by weight.

Mol % figures were determined by NMR spectroscopy and always relate, unless otherwise stated, to the sum of the NCO conversion products. The measurements were carried out on Bruker DPX 400 or DRX 700 instruments on about 5% (¹H NMR) or about 50% (¹³C NMR) samples in dry C₆D₆ at a frequency of 400 or 700 MHz (¹H NMR) or 100 or 176 MHz (¹³C NMR). The reference employed for the ppm scale was small amounts of tetramethylsilane in the solvent having a ¹H NMR chemical shift of 0 ppm. Alternatively, the C₆D₅H present in the solvent was used as a reference signal: ¹H-NMR chemical shift 7.15 ppm, ¹³C-NMR chemical shift 128.02 ppm. Data for the chemical shift of the compounds in question were taken from the literature (cf. D. Wendisch, H. Reiff and D. Dieterich, Die Angewandte Makromolekulare Chemie 141, 1986, 173-183 and literature cited therein and also EP 896 009 A1).

Dynamic viscosities were determined at 23° C. using a Haake VT 550 viscometer in accordance with DIN EN ISO 3219:1994-10. By measurements at different shear rates, it was ensured that the flow behavior of the polyisocyanate mixtures described according to the invention and also that of the comparative products corresponds to that of ideal Newtonian fluids. The indication of the shear rate can therefore be omitted.

The NCO content was determined by titration in accordance with DIN EN ISO 11909:2007-05.

The residual monomer contents were determined by gas chromatography in accordance with DIN EN ISO 10283:2007-11 using an internal standard.

All reactions were conducted under a nitrogen atmosphere unless stated otherwise.

The diisocyanates used are products of Covestro AG, D-51365 Leverkusen; all other commercially available chemicals were sourced from Aldrich, D-82018 Taufkirchen.

The reactants that were not commercially available were obtained by methods known from the literature.

### Example 1: Catalyst Production (Noninventive)

In a 2 l four-necked flask fitted with a stirrer and an intensive cooler, 61.7 g (1.1 mol) of KOH, 500 ml of water (deionized) and 139.7 g (1.1 mol) of 1,4-dichlorobutane were together initially charged and heated to an oil bath temperature of about 100-110° C. with stirring (gentle reflux).

After attaining the aforementioned internal temperature, 85.2 g (1 mol) of piperidine were added sufficiently quickly to avoid excessive reflux.

Solid separation already occurred shortly after commencement of piperidine addition. The reaction mixture was heterogeneous from the outset (initially liquid-liquid, then triphasic liquid-liquid-solid, then solid-liquid), but always contained sufficient liquid phase for efficient mixing of the majority of the reaction mixture.

After piperidine addition was complete the mixture was stirred for a further two hours at an oil bath temperature of 110-120° C. Reflux decreased noticeably over this period.

Subsequently about 150 g of methanol were added to the still-hot mixture and the latter was boiled under reflux until a finely divided suspension had formed. The mixture was then cooled to about 40° C. and about 87 g (1.5 mol) of solid KF were added and mixed with enough methanol (250-300 g, precise amount noncritical) to ensure good stirrability.

Subsequently at 40° C. and with occasional argentometric chloride monitoring the previously filtered liquid phase was stirred and then filtered. The filter residue was subjected to portionwise washing with a total of about 400 g of 2-ethylhexanol (2-EH) preheated to about 40-50° C. and the combined filtrates were admixed with 50 g of 40% aqueous hydrofluoric acid (1 mol HF) with stirring and cooling such that the internal temperature did not exceed 40° C.

Once HF addition was complete the mixture was heated to reflux for one hour and the apparatus then switched to distillate removal. Once no more distillate ran over at standard pressure and a bath temperature of 100° C., the pressure was reduced stepwise to 50 mbar, the bottoms temperature was increased stepwise to a maximum of 150° C. and the obtained distillate was discontinuously discharged. Finally anhydrous distillate (2-EH) was obtained overhead.

After cooling, the target concentration (20%) was established by 2-EH addition followed by filtration to remove small amounts of insoluble constituents. The total fluoride content of this clear, yellowish solution (ion-sensitive electrode; F from HF and fluoride from halogen exchange captured as total fluoride) was 4.3%. The F deriving from the HF addition was acidimetrically titrated (as H from HF) with 0.1 N NaOH against phenolphthalein and determined at 2.1% and the (argentometrically determined) residual chloride content was 0.15%.

The other catalysts specified in table 1 were correspondingly synthesized from the primarily formed chloride by anion exchange (optionally followed by HF addition for the synthesis of the di/polyfluorides). The monocyclic ammonium salts 5-7 were synthesized on the basis of commercially available N,N-dimethylammonium chloride, likewise as shown above, by anion exchange (optionally followed by HF addition for the synthesis of the di/polyfluorides).

TABLE 1
Overview of the catalysts produced (catalyst concentration relates to the active compound
(cation and anion))

| Catalyst | Cation | Anion | Concentration [%] |
|---|---|---|---|
| 1 5-azoniaspiro [4.4]nonanium hydroxide | 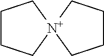 | OH⁻ | 1 |
| 2 5-azoniaspiro[4.5]decanium hydrogendifluoride | 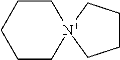 | [HF₂]⁻ | 20 |
| 3 6- azoniaspiro[5.5]undecanium pivalate | 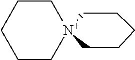 | (CH₃)₃CC(O)O⁻ | 10 |
| 4 6- azoniaspiro[5.5]undecanium hydrogendifluoride | 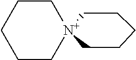 | [HF₂]⁻ | 20 |
| 5 1,1-dimethylpyrrolidin-1- ium hydroxide | 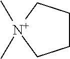 | OH⁻ | 1 |
| 6 1,1-dimethylpyrrolidin-1- ium hydrogendifluoride | 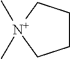 | [HF₂]⁻ | 10 |
| 7 1,1-dimethylpiperidin-1- ium pivalate | 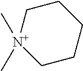 | (CH₃)₃CC(O)O⁻ | 10 |

### Example Series 2: Noninventive, Process According to Example Series 2-1 to 2-4 of EP 3107948 and Example Series 2-5 to 2-7 of EP 3337836

The optimal catalyst concentration for the diisocyanate trimerization was determined in exploratory preliminary tests at 60° C. and the concentration of the catalyst solution was adjusted by diluting with 2-EH such that only negligible gel particle formation, if any, was observable upon addition of the catalyst solution to the stirred HDJ. The test series with distillate recycling on a 1 kg scale was then performed as described below.

A double-walled flanged vessel heated to the starting temperature desired in each case using an external circuit and fitted with a stirrer, a reflux condenser connected to an inert gas system (nitrogen/vacuum) and a thermometer was initially charged with 1000 g of HDI which was freed of dissolved gases by stirring under reduced pressure (<1 mbar) for one hour. After gasification with nitrogen the catalyst type and amount specified in table 2 was added in the concentration specified in table 2 in such a way that the reaction was able to be run at a temperature range between about 60° C. and 65° C. After about 1 mol of NCO groups had been converted, as indicated by attainment of an NCO content of around 45.8%, the catalyst was deactivated by addition of an amount equivalent to the catalyst of the stopper specified in table 2 and the mixture was stirred at reaction temperature for a further 30 min and subsequently worked up.

Workup was carried out by vacuum distillation in a thin-film evaporator of the flash evaporator (FE) type with a preevaporator (PE) connected upstream (distillation data:pressure: 0.08+/−0.04 mbar, PE temperature: 120° C., ME temp.: 140° C.), wherein unconverted monomer was separated as distillate and the low-monomer polyisocyanate resin was separated as bottoms product (starting run). The polyisocyanate resin was separated and the distillate was collected in a second flanged stirring apparatus of identical construction to the first and made up to the starting amount (1000 g) with freshly degassed HDJ. Thereafter, the mixture was treated again with catalyst and the procedure as described at the outset was followed. This procedure was repeated multiple times (tests A, B, C, etc.). After multiple recycling steps it was observed that the catalyst amount required to achieve the desired conversion was initially slightly higher for the first test of each series than for the subsequent test but then increased successively. Furthermore, especially for the products of the “later” recycling steps (tests D et seq.) and often only after multiple weeks of storage and/or after addition of typical lacquer solvents such as xylene or solvent naphtha, slow-onset clouding was observable for the polyisocyanate resins, occasionally accompanied by a slow increase in the residual monomer content, especially during storage at relatively high temperature. According to the results of combined analytical methods the precipitates obtained after filtration proved to be essentially the ammonium chlorides (about ⅔ by mole) and bromides (about ⅓ by mole) of the employed catalysts. The results are apparent from table 2.

TABLE 2
Performed isocyanate modifications (comparative examples)

| | Catalyst as per | Catalyst soln. | |
|---|---|---|---|
| Example no. 2- . . . | table 1 | [g] | Stopper*⁾ |
| -1-A | 1 | 7.9 | 1 |
| -1-B | 1 | 6.1 | 1 |
| -1-C | 1 | 6.2 | 1 |
| -1-D | 1 | 6.4 | 1 |
| -1-E | 1 | 6.5 | 1 |
| -1-F | 1 | 7.1 | 1 |
| -2-A | 2 | 0.28 | 2 |
| -2-B | 2 | 0.20 | 2 |
| -2-C | 2 | 0.21 | 2 |
| -2-D | 2 | 0.25 | 2 |
| -2-E | 2 | 0.31 | 2 |
| -2-F | 2 | 0.45 | 2 |
| -3-A | 3 | 1.92 | 1 |
| -3-B | 3 | 1.70 | 1 |
| -3-C | 3 | 1.72 | 1 |
| -3-D | 3 | 1.85 | 1 |
| -3-E | 3 | 2.01 | 1 |
| -3-F | 3 | 2.94 | 1 |
| -4-A | 4 | 0.58 | 3 |
| -4-B | 4 | 0.52 | 3 |
| -4-C | 4 | 0.61 | 3 |
| -4-D | 4 | 0.68 | 3 |
| -4-E | 4 | 0.72 | 3 |
| -4-F | 4 | 0.89 | 3 |
| -5-A | 5 | 0.86 | 3 |
| -5-B | 5 | 0.90 | 3 |
| -5-C | 5 | 0.65 | 3 |
| -5-D | 5 | 1.24 | 3 |
| -5-E | 5 | 1.56 | 3 |
| -5-F | 5 | 1.80 | 3 |
| -6-A | 6 | 1.45 | 2 |
| -6-B | 6 | 0.92 | 2 |
| -6-C | 6 | 1.01 | 2 |
| -6-D | 6 | 1.25 | 2 |
| -6-E | 6 | 1.35 | 2 |
| -6-F | 6 | 1.52 | 2 |
| -7-A | 7 | 4.30 | 1 |
| -7-B | 7 | 3.98 | 1 |
| -7-C | 7 | 4.10 | 1 |
| -7-D | 7 | 4.69 | 1 |
| -7-E | 7 | 5.10 | 1 |
| -7-F | 7 | 5.26 | 1 |

*⁾Stopper: 1: di-n-butyl phosphate, 2: toluenesulfonic acid, 40% in 2-PrOH, 3: dodecylbenzenesulfonic acid, 70% in 2-PrOH

### Example Series 3: According to the Invention

A process as in example series 2 was specified with the exception that after achieving the target NCO content (about 45.8%) the catalyst was deactivated by addition of an amount of the stopper specified in table 3 which was substoichiometric relative to the catalyst (40-60 mol % based on the employed catalyst), the reaction mixture was stirred at reaction temperature for a further 30 min to ensure that the reaction was efficiently inhibited and the mixture was subsequently worked up as specified in example series 2.

The polyisocyanate resin isolated after each cycle was separated and subsequently post-stabilized with the amount missing from 110% of the stopper amount based stoichiometrically on employed catalyst by stirring for one hour at 60° C. after addition of the stabilizer. Even after multiple recycling steps no increase in the catalyst amount necessary for achieving the target conversion in the respective subsequent test was observable. Furthermore, none of the obtained products showed delayed-onset clouding in the polyisocyanate resins or an increase in the residual monomer content under the conditions recited in comparative example series 2.

The results are apparent from table 3.

TABLE 3
Isocyanate modifications performed (examples
according to the invention)

| | Catalyst as per | Catalyst soln. | |
|---|---|---|---|
| Example no. 2- . . . | table 1 | [g] | Stopper*⁾ |
| -1-A | 1 | 7.8 | 1 |
| -1-B | 1 | 6.0 | 1 |
| -1-C | 1 | 5.9 | 1 |
| -1-D | 1 | 6.0 | 1 |
| -1-E | 1 | 6.0 | 1 |
| -1-F | 1 | 6.0 | 1 |
| -2-A | 2 | 0.3 | 2 |
| -2-B | 2 | 0.2 | 2 |
| -2-C | 2 | 0.2 | 2 |
| -2-D | 2 | 0.2 | 2 |
| -2-E | 2 | 0.2 | 2 |
| -2-F | 2 | 0.2 | 2 |
| -3-A | 3 | 1.9 | 1 |
| -3-B | 3 | 1.7 | 1 |
| -3-C | 3 | 1.7 | 1 |
| -3-D | 3 | 1.6 | 1 |
| -3-E | 3 | 1.6 | 1 |
| -3-F | 3 | 1.6 | 1 |
| -4-A | 4 | 0.61 | 3 |
| -4-B | 4 | 0.50 | 3 |
| -4-C | 4 | 0.52 | 3 |
| -4-D | 4 | 0.51 | 3 |
| -4-E | 4 | 0.50 | 3 |
| -4-F | 4 | 0.50 | 3 |
| -5-A | 5 | 0.91 | 3 |
| -5-B | 5 | 0.85 | 3 |
| -5-C | 5 | 0.80 | 3 |
| -5-D | 5 | 0.81 | 3 |
| -5-E | 5 | 0.80 | 3 |
| -5-F | 5 | 0.81 | 3 |
| -6-A | 6 | 1.45 | 2 |
| -6-B | 6 | 1.0 | 2 |
| -6-C | 6 | 1.0 | 2 |
| -6-D | 6 | 1.0 | 2 |
| -6-E | 6 | 1.0 | 2 |
| -6-F | 6 | 1.0 | 2 |
| -7-A | 7 | 4.3 | 1 |
| -7-B | 7 | 3.9 | 1 |
| -7-C | 7 | 3.85 | 1 |
| -7-D | 7 | 3.8 | 1 |
| -7-E | 7 | 3.8 | 1 |
| -7-F | 7 | 3.75 | 1 |

*⁾Stopper/stabilizer: 1: di-n-butyl phosphate, 2: toluenesulfonic acid, 40% in 2-PrOH, 3: dodecylbenzenesulfonic acid, 70% in 2-PrOH

### Example Series 4: According to the Invention

A process as in example series 3 was specified with the exception that PDI (1,5-pentamethylenediisocyanate) was used instead of HDJ and after achieving the target NCO content (54.9% to 55.1%) the catalyst was deactivated by addition of an amount of the stopper specified in table 4 which was substoichiometric relative to the catalyst (40-60 mol % based on the employed catalyst) as described in example series 3, the reaction mixture was stirred at reaction temperature for a further 30 min to ensure that the reaction was efficiently inhibited and the mixture was subsequently worked up as specified in example series 2/3.

The polyisocyanate resin isolated after each cycle was separated and subsequently post-stabilized with the amount missing from 110% of the stopper amount based stoichiometrically on employed catalyst by stirring for one hour at 60° C. after addition of the stabilizer. Even after multiple recycling steps no increase in the catalyst amount necessary for achieving the target conversion in the respective subsequent test was observable. Furthermore, none of the obtained products showed delayed-onset clouding in the polyisocyanate resins or an increase in the residual monomer content under the conditions recited in comparative example series 2.

The results are apparent from table 4.

TABLE 4
Isocyanate modifications performed (examples
according to the invention)

| | Catalyst as per | Catalyst soln. | |
|---|---|---|---|
| Example no. 4- . . . | table 1 | [g] | Stopper*⁾ |
| -1-A | 1 | 8.2 | 1 |
| -1-B | 1 | 8.0 | 1 |
| -1-C | 1 | 8.0 | 1 |
| -1-D | 1 | 7.9 | 1 |
| -1-E | 1 | 7.8 | 1 |
| -1-F | 1 | 7.7 | 1 |
| -2-A | 2 | 0.5 | 2 |
| -2-B | 2 | 0.4 | 2 |
| -2-C | 2 | 0.3 | 2 |
| -2-D | 2 | 0.3 | 2 |
| -2-E | 2 | 0.3 | 2 |
| -2-F | 2 | 0.3 | 2 |
| -3-A | 3 | 2.5 | 1 |
| -3-B | 3 | 2.4 | 1 |
| -3-C | 3 | 2.4 | 1 |
| -3-D | 3 | 2.3 | 1 |
| -3-E | 3 | 2.3 | 1 |
| -3-F | 3 | 2.2 | 1 |
| -4-A | 4 | 0.8 | 3 |
| -4-B | 4 | 0.7 | 3 |
| -4-C | 4 | 0.7 | 3 |
| -4-D | 4 | 0.6 | 3 |
| -4-E | 4 | 0.5 | 3 |
| -4-F | 4 | 0.4 | 3 |
| -5-A | 5 | 1.2 | 3 |
| -5-B | 5 | 1.1 | 3 |
| -5-C | 5 | 1.1 | 3 |
| -5-D | 5 | 1.0 | 3 |
| -5-E | 5 | 0.9 | 3 |
| -5-F | 5 | 0.9 | 3 |
| -6-A | 6 | 1.7 | 2 |
| -6-B | 6 | 1.6 | 2 |
| -6-C | 6 | 1.5 | 2 |
| -6-D | 6 | 1.5 | 2 |
| -6-E | 6 | 1.4 | 2 |
| -6-F | 6 | 1.4 | 2 |
| -7-A | 7 | 4.5 | 1 |
| -7-B | 7 | 4.2 | 1 |
| -7-C | 7 | 4.1 | 1 |
| -7-D | 7 | 4.1 | 1 |
| -7-E | 7 | 4.0 | 1 |
| -7-F | 7 | 4.0 | 1 |

*⁾Stopper/stabilizer: 1: di-n-butyl phosphate, 2: toluenesulfonic acid, 40% in 2-PrOH, 3: dodecylbenzenesulfonic acid, 70% in 2-PrOH

1. A process for producing modified isocyanates where at least one organic di- and/or triisocyanate is reacted in at least the temporary presence of a component A1 comprising at least one tetramethylammonium salt and/or at least one cyclic ammonium salt having a cation of formula I 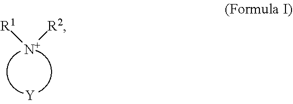
wherein
Y is a linear or branched C₂-C₂₀ segment optionally bearing further substituents and optionally interrupted by heteroatoms from the group of oxygen, sulfur, nitrogen and aromatic rings and optionally comprising further rings and
R¹ and R² are nitrogen substituents either independently represent identical or different, substituted or unsubstituted, optionally branched aliphatic C₁-C₂₀ radicals, aromatic C₆-C₂₀ radicals or araliphatic C₇-C₂₀ radicals or
the nitrogen substituents R¹ and R² together form a ring segment X for which the same or a different definition for Y mentioned above applies,
wherein the reaction is terminated upon achieving a predeterminable degree of conversion based on the total amount of NCO groups of the at least one organic di- and/or triisocyanate by addition of a substoichiometric amount, based on the molar amount of the cation of formula I in component A1, of a component A2 comprising at least one acidic compound which has a pKₐ value below 4.0 and is distinct from HF
and wherein the obtained modified isocyanate after optional purification is admixed with a component A3 comprising at least one acidic compound which has a pKₐ value below 4.0 and is distinct from HF, and
wherein the at least one acidic compound in component A2 and A3 may be identical or different.

2. The process of claim 1, wherein R¹ and R² in the cation of formula I independently represent identical or different C₁-C₈-alkyl substituents or identical or different benzyl radicals optionally substituted at the aromatic ring.

3. The process of claim 1, wherein Y in the cation of formula I represents an alkylene chain segment which comprises four to seven members together with the charge-carrying nitrogen atom and optionally bears further substituents.

4. The process of claim 1, wherein component A1 comprises an anion selected from the group consisting of hydroxide, alkanoate, carboxylate, heterocycles having at least one negatively charged nitrogen atom in the ring, fluoride, hydrogendifluoride, higher polyfluorides, adducts of more than one equivalent of HF onto compounds containing fluoride ions and any desired-mixtures thereof.

5. The process of claim 1, wherein the at least one acidic compound in component A1 and/or A2 has a pKₐ value of below 2.0.

6. The process of claim 1, wherein component A3 is added in a molar amount which together with component A2 is stoichiometric, based on the amount of the cation of formula I in component A1.

7. The process of claim 1, wherein the at least one acidic compound of component A2 and/or A3 is selected from the group consisting of alkanesulfonic and arylsulfonic acids.

8. The process of claim 1, wherein the organic diisocyanate is selected from the group consisting of PDI, HDI, MPDI, TMDI, NTI, IPDI, IMCI, XDI, H6XDI, MDI, TDI and NBDI.

9-11. (canceled)

12. A modified isocyanate produced, by the process of claim 1.

13. A one-component system comprising the modified isocyanate of claim 12 in which the NCO groups are blocked.

14. A coating produced by applying a one component system of claim 13 to a substrate and curing, optionally with heating and/or in the presence of a catalyst.

15. A composite component comprising a material that is at least partly bonded to a coating of claim 14.

16. The process of claim 1, wherein R¹ and R² in the cation of formula I independently represent methyl, or that R¹ and R² together with the charge-carrying nitrogen atom represent a ring segment X identical to Y or different from Y, wherein X is a C₄-C₆-alkylene chain segment optionally bearing further substituents and may optionally bear further substituents.

17. The process of claim 1, wherein Y in the cation of formula I represents an alkylene chain segment which comprises four to seven members together with the charge-carrying nitrogen atom and optionally bears further substituents.

18. The process of claim 1, wherein the at least one acidic compound of component A2 and/or A3 is an aromatic sulfonic acid.

19. The process of claim 1, wherein the organic diisocyanate is selected from the group consisting of pentamethylene diisocyanate, hexamethylene diisocyanate, isophorone diisocyanate, 1,3- and 1,4-bis(isocyanatomethyl)cyclohexane and norbornane diisocyanate.

20. A two-component system comprising a component 1) the modified isocyanate of claim 12 and a component 2) at least one NCO-reactive compound.

21. A coating produced by applying the two-component system of claim 20 to a substrate and curing, optionally with heating and/or in the presence of a catalyst.

22. A composite component comprising a material that is at least partly bonded to the coating of claim 21.
